(19) United States
(12) Patent Application Publication
(10) Pub. No.: US 2026/0241128 A1
(43) Pub. Date: Aug. 20, 2026

(54) Tracheal Intubation Balloon Cuff Through Which Fluid Passage Ducts Extend

(71) Applicants: ASSISTANCE PUBLIQUE - HOPITAUX DE PARIS, Paris (FR); ECOLE SUPERIEURE DE PHYSIQUE ET DE CHIMIE INDUSTRIELLES DE LA VILLE DE PARIS, Paris (FR); CENTRE NATIONAL DE LA RECHERCHE SCIENTIFIQUE (CNRS), Paris (FR); SORBONNE UNIVERSITE, Paris (FR); UNIVERSITE DE VERSAILLES-ST QUENTIN EN YVELINES, VERSAILLES CEDEX (FR)

(72) Inventors: Jean BERGOUNIOUX, Kremlin Bicêtre (FR); Emmanuel SIEFERT, Ixelles (BE); José BICO, PARIS (FR); Etienne REYSSAT, ANTONY (FR); Benoit ROMAN, Ivry-sur-Seine (FR)

(21) Appl. No.: 19/484,523

(22) PCT Filed: May 15, 2024
(86) PCT No.: PCT/EP2024/063442

§ 371 Date: Nov. 14, 2025

(30) Foreign Application Priority Data
May 16, 2023 (FR) FR2304872

(51) Int. Cl. A61M 16/04; A61M 16/00

(52) U.S. Cl. CPC A61M 16/044; A61M 16/0003; A61M 16/0402; A61M 2016/0027; A61M 2205/3327; A61M 2205/3344; A61M 2205/50; A61M 2210/1032; A61M 2230/40

(57) ABSTRACT

The invention relates to a balloon cuff (1) for a tracheal intubation device, the balloon cuff (1) being elastically deformable and extending between an inner surface (3) surrounding an axis (A) and an outer surface (5) surrounding the inner surface (3), the balloon cuff (1) comprising inner walls that define ducts (13, 15) that extend through the balloon cuff, the balloon cuff (1) being configured to go from a deflated state to an inflated state by injecting a fluid into the ducts (13, 15) such that the outer surface (5) moves away from the axis.

### FIELD OF THE INVENTION

The present invention belongs to the technical field of medical devices for creating and maintaining an artificial respiration pathway in the trachea of a patient, especially for invasive mechanical ventilation (IMV).

The invention more specifically concerns an improved balloon cuff for a tracheal intubation device, for example for an endotracheal intubation tube or for a tracheostomy or stoma cannula. The invention also concerns a tracheal intubation device comprising such a balloon cuff and a method for monitoring an inflation state of such a balloon cuff.

### STATE OF THE ART

Mechanical ventilation is a widespread and effective solution for artificially supplying oxygen to patients with respiratory failure, and for removing carbon dioxide released by the respiratory system. Other objectives of mechanical ventilation are to reduce respiratory effort for the ventilated patient, and to treat potentially fatal conditions for the patient such as hypoxemia, hypercapnia, insufficient arterial blood oxygenation, and acute respiratory acidosis.

The term “invasive mechanical ventilation” (IMV) is commonly used to designate mechanical ventilation in which an artificial airway device is inserted into the patient's body (as opposed to non-invasive ventilation, which usually uses a respiratory mask). Invasive mechanical ventilation is especially used in acute or chronic respiratory failure, or to maintain the functionality of the patient's airway during a procedure, such as, for example, during intubation in the intensive care unit or in the operating room.

There are several types of instruments providing an artificial airway at the trachea, for connection to a mechanical respirator in order to carry out gas exchanges.

The endotracheal tube is intended to be inserted through the mouth or nose of the patient into the trachea, without necessarily performing a surgical procedure. Such a tube is mainly used in adult or pediatric patients with acute respiratory failure. To ensure the sealing of the distal airways, i.e., to prevent communication of fluid, air or liquid, between these distal airways and the upper airways (especially the pharynx and larynx), the endotracheal tube is often equipped with a balloon cuff placed along the tube. Airway sealing is ensured when the balloon cuff is inflated and brought into contact with the trachea.

Such a balloon cuff makes it possible to isolate the tracheobronchial tree fluidly and bacteriologically. A balloon cuff is especially recommended to protect the patient's lungs from reflux and contamination by oral and digestive secretions. This balloon cuff provides airway sealing and prevents air leaks around the intubation tube, allowing control of the volumes and pressures that are administered to the patient. This pressure/volume regime is fundamental in the treatment of patient respiratory failure regardless of age or causal pathology.

The tracheostomy cannula, on the other hand, is inserted not through the patient's natural airways, but through a stoma at the trachea (surgical opening, opening the trachea to the outside). This solution is particularly suitable for patients suffering from chronic respiratory failure, for whom mechanical ventilation is implemented in the long term. Tracheotomy cannulas are also commonly equipped with a balloon cuff for the same reasons mentioned above in relation to conventional intubation tubes.

Invasive mechanical ventilation can greatly improve the life expectancy of patients with acute respiratory failure.

Invasive mechanical ventilation is, however, linked to a hospital mortality rate greater than 35% for non-surgical patients undergoing invasive mechanical ventilation. In addition, there is significant long-term morbidity for patients allowed to return home after the respiratory failure event.

This significant mortality is partly related to two shortcomings of current tracheal intubation balloon cuffs.

On the one hand, micro-inhalations of contaminated oropharyngeal secretions can cause bronchopneumopathies (known as “ventilator acquired pneumonia”, or VAP). Complications of invasive mechanical ventilation are considered to be more than 30% attributable to such acquired bronchopneumopathies. These leaks of oral secretions to the tracheobronchial system and deep lung are due to insufficient sealing of the tracheal intubation balloon cuff. Insufficient sealing is caused by undesirable folds in the outer surface of the balloon cuff, creating micro-channels through which oral secretions, i.e. oropharyngeal secretions on the “mouth” side of the balloon cuff, can pass. The phenomenon of folding of the outer surface of the balloon cuff is especially described in the publication “La ventilation mécanique, de la physiologie à la pratique: quelle sonde, quel circuit, quel ventilateur? [Mechanical ventilation, from physiology to practice: which tube, which circuit, which ventilator?],” Anesthésie & Réanimation, Volume 4, Issue 2, March 2018, pp. 180-189 (see FIG. 1 of this document, page 182).

A known solution to overcome the insufficient sealing of the balloon cuff is to use additional suction systems that collect oral secretions above the proximal end of the balloon cuff. However, such suction systems cause additional pain for patients and require maintenance.

On the other hand, excessive pressure exerted by the outer surface of the balloon cuff on the tracheal wall can cause severe damage to the tracheal mucosa and submucosa. Tracheal damage due to hyperinflation of the balloon cuff can impair blood circulation to the tracheal mucosa, causing pressure wounds and possible complications such as necrosis or, in case of scarring, tracheal stenosis. A stenosis is a particular scarring of the trachea that reduces its diameter and can complicate breathing.

The usual solution for maintaining optimal balloon cuff pressure, sufficient to maintain seal of the distal airways while being limited so as not to cause tracheal damage, is to manually monitor and adjust the air pressure inside the balloon cuff. It is estimated that, to maintain balloon cuff pressure within the recommended ranges, up to eight daily manual balloon cuff pressure adjustments are required. However, the need for frequent adjustments increases the workload of hospital staff, and there is a risk of human error.

More recent systems exist, including, for example, the NOSTEN® system, which allow better control of the balloon cuff pressure, and especially adaptive control of this pressure. However, these systems use standard balloon cuffs whose sealing is insufficient. These systems therefore do not completely prevent leakage of oral secretions to the tracheobronchial system and the lungs.

It should be noted that when invasive ventilation is implemented, the patient's tracheobronchial system is put in closed circuit with the ventilation machine. The tracheobronchial system becomes isolated from the external environment. Bronchial secretions on the lung side of the balloon cuff cannot be eliminated toward the external environment, for example, be swallowed by clearing the throat. It is then essential to carry out regular tracheal aspiration—at least every four hours—which consists of opening the circuit, sliding a probe into the throat of the patient and aspirating the accumulated secretions. This procedure is invasive and damaging to the trachea. In addition, the intubated patient who is normally ventilated with an excess pressure on the lung side—even at the end of expiration—to maximize the alveolar exchanges then experiences a vacuum which degrades the alveolar exchanges and the quality of the patient's breathing.

### DISCLOSURE OF THE INVENTION

In view of the above, there is a need for a balloon cuff device for tracheal intubation that is improved with regard to the existing cuffs; reducing the risk of tracheal injuries in the patient, especially during mechanical ventilation, while ensuring a satisfactory seal between the patient's distal airways and upper airways.

Preferably, the tracheal intubation device sought comprises a balloon cuff whose pressure on the trachea is easily adjustable, so as to reduce the workload of hospital staff during mechanical ventilation.

There is an additional need for a tracheal intubation balloon cuff whose radial pressure adapts according to certain patient movements and actions, for example in response to a coughing or swallowing effort by the patient.

A tracheal intubation device is also sought that is capable of autonomously expelling the patient's oropharyngeal secretions, so as to avoid contamination of the distal respiratory tract by said secretions and to reduce or eliminate the need for tracheal aspiration, i.e., aspiration of bronchial secretions.

In order to meet these needs, a first aspect of the invention relates to a balloon cuff for a tracheal intubation device, the balloon cuff being elastically deformable and extending between an inner surface surrounding an axis and an outer surface surrounding the inner surface, the balloon cuff comprising inner walls defining ducts passing through the balloon cuff, the balloon cuff being configured to transition from a deflated state to an inflated state by injection of a fluid into the ducts so that the outer surface moves away from the axis.

Such a balloon cuff is advantageously and optionally supplemented by the following different characteristics taken alone or in combination:
a ratio of a thickness, when the balloon cuff is in the deflated state, between the inner surface and the outer surface over a width of the outer surface is greater than 10%, preferably 20%;
for each duct, a ratio of a duct size, when the balloon cuff is in the inflated state, over a closer distance between the duct and the outer surface is less than or equal to 100%, preferably 50%;
the inner surface and the outer surface have cylindrical shapes defined by an axial direction parallel to the axis, the inner surface and the outer surface having at least partly circular cross-sections in a plane orthogonal to the axis, preferably the outer surface has a flat area extending along the entire outer surface in the axial direction in a plane parallel to the axis;
the ducts have a main direction parallel to the axis, the ducts preferably extending parallel to the axis from a proximal end of the balloon cuff to a distal end of the balloon cuff;
the ducts have a main direction parallel to a circumferential direction with regard to the axis, the ducts preferably extending in an arc of a circle around the axis;
the balloon cuff comprises different groups of independently controllable ducts, the groups being located in sections of the balloon cuff, the sections being contiguous in pairs and placed successively along the axis;
the thickness between the inner surface and the outer surface is less than or equal to 1 mm, preferably 0.5 mm, when the balloon cuff is configured in the deflated state; and
the balloon cuff is formed predominantly of synthetic polymer material, preferably of silicone or polyurethane.

The invention also relates to a tracheal intubation device configured to form an artificial airway at the patient's trachea, the intubation device comprising:
an artificial respiration tube,
a balloon cuff as just presented, the balloon cuff being mounted on the artificial respiration tube, so that the inner surface surrounds the tube.

Such a device advantageously and optionally comprises a pressure sensor downstream of the balloon cuff with regard to the cranio-caudal direction, the sensor being configured to acquire a pressure measurement representative of a coughing effort by the patient.

Finally, the invention covers a method of controlling such a tracheal intubation device, the method comprising setting the outer surface in motion by injecting fluid inside a first group of ducts and by injecting fluid inside a second group of ducts, so as to modify a state of inflation of the outer surface.

Such a method advantageously and optionally comprises a detection of an excess pressure in the trachea, the movement being triggered as a function of the excess pressure detected, when the device comprises a pressure sensor downstream of the balloon cuff with regard to the cranio-caudal direction, the sensor being configured to acquire a pressure measurement representative of a coughing effort by the patient.

### DESCRIPTION OF THE FIGURES

Other characteristics and advantages of the invention will emerge from the following description, which is purely illustrative and non-limiting, and which must be read with reference to the attached drawings in which:

FIGS. 1 to 3 are schematic representations of a balloon cuff according to different embodiments of the invention;

FIG. 4 is a schematic representation of a tracheal intubation device according to an embodiment of the invention 1; and

FIG. 5 is a schematic representation of a movement of the outer surface of the balloon cuff.

### DETAILED DESCRIPTION OF THE INVENTION

### Balloon Cuff for a Tracheal Intubation Device

According to a first aspect, the invention relates to a balloon cuff 1 for a tracheal intubation device. The balloon cuff 1 is configured to be mounted on an artificial respiration tube of the tracheal intubation device.

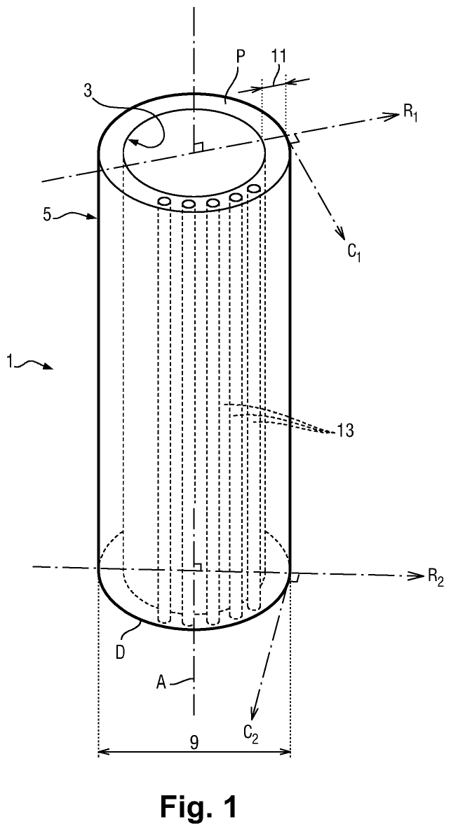

With reference to FIGS. 1 to 3, a balloon cuff 1 extends along an axis A, or central axis, between a proximal end P and a distal end D.

When the balloon cuff 1 is slid over a tube of an intubation device, the central axis of the balloon cuff corresponds to the axis of the tube.

When the balloon cuff, mounted on the tube, is inserted into the patient's trachea the central axis corresponds to the general direction of extension of the trachea, also known as the “cranio-caudal” direction.

The proximal end corresponds to the “mouth side” of the balloon cuff.

The distal end corresponds to the “bronchial or lung side” of the balloon cuff.

Radial directions and circumferential directions are defined from the central axis.

For example, in FIG. 1, there is shown at the proximal end P of the balloon cuff a radial direction R1 orthogonal to the central axis A and a circumferential direction C1 orthogonal to the radial direction R1 and to the central axis A. Likewise, in FIG. 1, there is shown at the distal end D of the balloon cuff a radial direction R2 orthogonal to the central axis A and a circumferential direction C2 orthogonal to the radial direction R2 and to the central axis A.

The balloon cuff extends radially with regard to the central axis from an inner surface 3 to an outer surface 5. The inner surface 3 surrounds the central axis A. The outer surface 5 surrounds the inner surface 3.

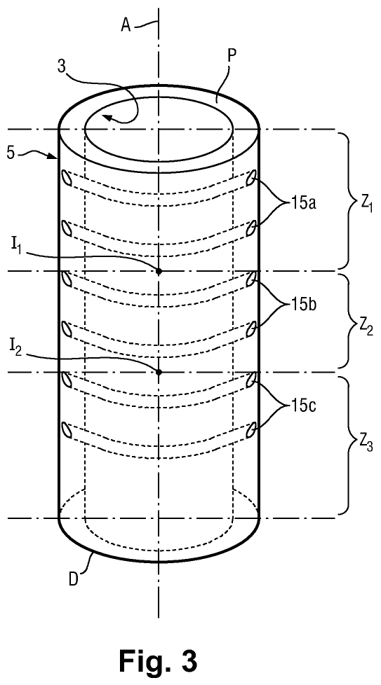

For example, and as shown in FIGS. 1 and 3, the outer surface 5 and the inner surface 3 are two cylinders of circular cross-section whose axis is the central axis. The balloon cuff 1 then has the shape of a hollow tube which has, in a radial plane, i.e., orthogonal to the central axis A, a section in the form of a circular ring.

A thickness 11 of the balloon cuff is defined as the distance in a radial direction between the inner surface 3 and the outer surface 5. With reference to FIG. 1, the thickness 11 is, for example, defined along the radial direction R1 at the proximal end P of the balloon cuff. When the outer surface 5 and the inner surface 3 are two cylinders of circular cross-section whose axis is the central axis, the thickness 11 is the radial thickness of the circular ring.

A width 9 of the outer surface is defined as the distance along a radial direction between the two points of intersection of the outer surface and the radial direction. The two intersection points are separated by the central axis. With reference to FIG. 1, the width 9 is, for example, defined in the radial direction R2 at the distal end D of the balloon cuff. When the outer surface 5 and the inner surface 3 are two cylinders of circular cross-section whose axis is the central axis, the width of the outer surface is the outer diameter of the circular ring.

The balloon cuff 1 is elastically deformable.

Advantageously, the balloon cuff has an elastic deformation rate greater than or equal to 10%.

More advantageously, the balloon cuff has an elastic deformation rate greater than or equal to 20%.

Even more advantageously, the balloon cuff has an elastic deformation rate greater than or equal to 30%.

An elastic deformation rate of an object between a neutral state and a deformed state is defined here as a ratio of the difference between a length of the object in the deformed state and a length of the object in the neutral state to the length of the object in the neutral state. The deformation is elastic if the object in the deformed state can reversibly return to the neutral state, and especially without breaking or plastic deformation of the object.

In particular, the balloon cuff may be made of elastomer, especially polyurethane, silicone, and mixtures thereof.

Preferably, the balloon cuff may be formed predominantly, i.e., more than 50% by mass, of synthetic polymer material, preferably silicone.

The balloon cuff 1 comprises inner walls defining ducts 13, 15 or channels that extend through the balloon cuff.

The balloon cuff comprises, for example, a total number of ducts comprised between 4 and 20, preferably between 8 and 12, but their number may vary more or less.

Each duct extends into the balloon cuff 1 between the inner surface 3 and the outer surface 5. Each duct extends in a main direction so that the dimension of the duct in the main direction is significantly larger than in the other directions. A cross-section of the duct can be defined at any point of the duct as the intersection between the duct and a plane orthogonal to the main direction at that point.

The cross-section corresponds to a flat surface which has a perimeter closed on itself, which perimeter is curved or formed by different rectilinear segments.

The size of a duct is a characteristic size of the duct cross-section; it can be defined, for example, as the diameter of the largest sphere that can be inserted into the duct without deformation.

The size of the duct is therefore the transverse dimension of the duct in a plane orthogonal to the main direction of the duct.

The main direction can be constant and in this case the duct is straight. The main direction can vary continuously along the duct and in this case the duct is curved. The duct may also comprise straight portions and curved portions.

The balloon cuff is configured to transition from a deflated state to an inflated state by injecting fluid into each duct so that the outer surface moves away from the axis, i.e., moves radially toward the exterior of the balloon cuff.

When the fluid is injected into a duct, the duct is pressurized so that the cross-section of the duct increases.

When fluid is no longer injected into the ducts, the balloon cuff goes from the inflated state to the deflated state.

The transformation of the balloon cuff during the injection of fluid into the ducts is reversible. In other words, the balloon cuff returns to its initial deflated state once the fluid has been removed.

Calibrating the fluid injection produces balloon cuff deformations that are calibrated, predictable and reproducible.

The fluid injected into the ducts of the balloon cuff to obtain this type of transformation may be liquid, gas or a mixture of liquid and gas. Different liquids or gases may be used alone or in combination.

Preferably, the fluid is
either air (this is called pneumatic operation),
or water (this is called hydraulic operation).

To inject the fluid, it is possible to control a fluid injection rate and/or pressure and/or fluid injection volume.

The shape of the balloon cuff can be controlled by injecting a fluid (gas or liquid) into the ducts that pass through it. In this case, the balloon cuff can go from a deflated state to an inflated state, with the outer surface of the balloon cuff moving radially outward. Such a balloon cuff inserted into the patient's trachea in a deflated state may then be inflated with the outer surface contacting the trachea. The presence of several ducts makes it possible to distribute and homogenize the areas of inflation in the balloon cuff. Such a balloon cuff provides a seal between the distal airways and the upper airways of the patient that is better than in the prior art.

According to a first option, it is possible to require that the ratio of the thickness, when the balloon cuff is in the deflated state, between the inner surface 3 and the outer surface 5 over the width 9 of the outer surface 5 is greater than 10%, and preferably 20%.

Such a ratio makes it possible to reduce the risk of folds in the outer surface during inflation of the balloon cuff.

According to a second option, optionally combined with the first option, it is possible for each duct to require that the size of the duct 13, 15 is less than the closest distance between the duct 13, 15 and the outer surface 5. More precisely, it is possible to require that the ratio between the size of the duct 13, 15 and the closest distance between the duct 13, 15 and the outer surface 5 is less than or equal to 100%, and preferably 50%.

Such a ratio makes it possible to limit the formation of bulges and bumps in the outer surface with regard to the duct during the inflation of this duct. The sealing of the balloon cuff is thus improved compared with the prior art.

The deflated state allows the balloon cuff surrounding the tube to slide into the trachea.

The inflated state brings the outer surface of the balloon cuff into contact with the trachea, with the balloon cuff surrounding the tube in the trachea. The contact between the outer surface of the balloon cuff and the trachea is more homogeneous than in the prior art. The pressure of the balloon cuff on the trachea is applied more homogeneously than in the prior art.

It should be noted that in this case the inner surface is facing the tube. The distance between the central axis and the inner surface remains greater than or equal to the radius of the tube so that the diameter of the tube is not reduced by the action of the balloon cuff.

For this purpose, the tube may be significantly stiffer than the balloon cuff, or the balloon cuff may be configured so that, regardless of the configuration of the balloon cuff, the distance between the central axis and the inner surface always remains equal to, or slightly greater than, the radius of the tube.

The balloon cuff presented above can also find a possible application in the placement of coronary, arterial, cardiac or venous stents in the fields of interventional radiology or cardiology. The fact that the pressure is applied more homogeneously to the balloon cuff surface is a very relevant element for this type of application.

Preferably, the thickness between the inner surface and the outer surface may be chosen to be less than or equal to 2 millimeters, or 1 millimeter or 0.5 millimeters, when the balloon cuff is configured in the deflated state. Conventionally, a tube has a diameter of less than or equal to 1 millimeter. The “tube+deflated balloon cuff” assembly has an outer width which depends on the thickness between the inner surface and the outer surface: This outer width of the “tube+deflated balloon cuff” assembly may take a value of less than 5 millimeters, or 3 millimeters or 2 millimeters.

### Balloon Cuff Geometries for a Tracheal Intubation Device

The inner surface 3 and the outer surface 5 may have different shapes. In a non-limiting manner, the shape of these surfaces may be:
cylindrical defined by an axial direction parallel to the axis, so that the inner and outer surfaces have circular cross-sections in a plane orthogonal to the axis, or
cylindrical defined by the axial direction, so that the inner and outer surfaces have elliptical sections in a plane orthogonal to the axis, or else
cylindrical defined by the axial direction, so that the inner surface and the outer surface have a section with a flat area in a plane orthogonal to the axis.

In a preferred embodiment, the outer surface 5 and the inner surface 3 are two cylinders of circular cross-section whose axis is the central axis. In other words, the inner surface and the outer surface have cylindrical shapes defined by the axial direction, the inner surface and the outer surface having circular cross-sections in a plane orthogonal to the axis.

The balloon cuff 1 then has the shape of a hollow tube which has, in a radial plane, i.e., orthogonal to the central axis A, a section in the form of a circular ring.

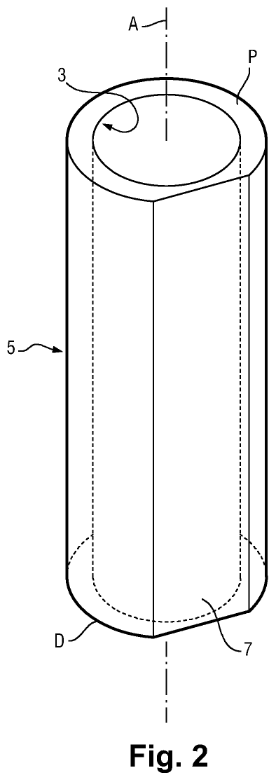

Advantageously, in this preferred embodiment, and with reference to FIG. 2, the outer surface has a flat area 7 which extends along the entire outer surface in the axial direction in a plane parallel to the axis. The flat area 7 defines a flat surface parallel to the axis and orthogonal to a direction radial to the axis. The shortest distance separating the flat area 7 and the axis is greater than the radius of the inner surface 3, so that the inner surface 3 is located between the outer surface 5 and the axis A.

The flat area 7 corresponds to an angular extent in a radial plane orthogonal to the axis advantageously comprised between 20° and 100°, and even more advantageously between 40° and 80°.

A balloon cuff comprising such a flat area has a shape more adapted to the anatomy of the trachea so that insertion of the balloon cuff into the trachea and inflation is less traumatic for the patient.

### Geometries of the Network of Ducts in the Balloon Cuff

Regardless of the geometry of the inner surface and the outer surface of the balloon cuff, the ducts may take different shapes between these two surfaces.

In a first embodiment of the ducts of the balloon cuff, and with reference to FIG. 1, at least part of the ducts 13 have a main direction parallel to the axis, i.e., the dimension of the duct along the axis is significantly greater than the dimension of the duct along the other directions. These ducts preferably extend parallel to the axis from the proximal end P of the balloon cuff to the distal end D of the balloon cuff. When these ducts are pressurized, the technical effect associated with this characteristic is to achieve inflation of the balloon cuff in the circumferential direction rather than in the axial direction, which induces expansion of the balloon cuff section.

As an option of this first embodiment of the ducts of the balloon cuff, the ducts 13 which extend parallel to the axis are configured so that, when the balloon cuff is in the deflated state, for each group of two adjacent ducts, an angular distance in a radial plane between the two adjacent ducts is equal to a predetermined distance. In other words, the angular difference in a radial plane that separates two adjacent ducts is constant around the axis.

In a second embodiment of the ducts of the balloon cuff, and with reference to FIG. 3, at least a portion of the ducts have a main direction parallel to a circumferential direction—or equivalently orthoradially—with regard to the axis, i.e. the dimension of the duct in the circumferential direction is significantly larger than the dimension of the duct in the other directions, especially the axial direction and the radial direction. These ducts 15a, 15b and 15c extend perpendicularly to the axis, preferably in an arc of a circle around the axis. When these ducts are pressurized, the technical effect associated with this characteristic is to achieve inflation of the balloon cuff in the axial direction, rather than in the circumferential direction.

As an option of this second embodiment of the balloon cuff ducts, the ducts 15a, 15b and 15c which extend parallel to the circumferential direction are configured so that, when the balloon cuff is in the deflated state, for each group of two adjacent ducts an axial distance along the axis is equal to a predetermined distance. In other words, the axial distance along the axis separating two adjacent ducts is constant along the axis.

The two duct modes presented here are compatible with each other.

In particular, the ducts can be separated into at least two groups:
a first group of ducts having a main direction parallel to the axis, and
a second group of ducts having a main direction parallel to the circumferential direction.

These two groups can be independently controlled regarding their pressurization by the injected fluid. In other words, the fluid can be injected into the ducts of a first group without injecting the fluid into the ducts of the second group.

The first group can especially ensure the deployment of the balloon cuff and the sealing between the upstream and downstream of the balloon cuff.

More generally, the ducts can be organized into different groups that can be controlled independently regarding their pressurization by the injected fluid.

In particular, independently controllable groups can be organized into sections of the balloon cuff placed successively along the axis of the balloon cuff. The groups are located in sections of the balloon cuff, the sections being contiguous in pairs and placed successively along the axis.

For example, and with reference to FIG. 3, the balloon cuff may comprise a second group of ducts having a main direction parallel to the circumferential direction, the second group being formed of three subgroups 15a, 15b and 15c of ducts placed in three different axial zones defined by:
the proximal end P,
a first intermediate point I1 located axially between the proximal end P and distal end D,
a second intermediate point I2 located axially between the first intermediate point I1 and the distal end D, and
the distal end D.

The first zone of the balloon cuff Z1, or proximal zone, is defined between the proximal end P and the first intermediate point I1. The second zone Z2 of the balloon cuff, or central zone, is defined between the two intermediate points I1 and I2. The third zone of the balloon cuff Z3, or distal zone, is defined between the second intermediate point I2 and the distal end D.

In this case, all the ducts in a subgroup are located in one of the zones and each zone comprises only one of the three subgroups of ducts, here:
a proximal subgroup of ducts 15a in the proximal zone Z1,
a central subgroup of ducts 15b in the central zone Z2, and
a distal subgroup of ducts 15c in the distal zone Z3.

More generally, the balloon cuff may comprise a number n greater than 3 subgroups of ducts in a number n of zones.

Still more generally, the balloon cuff may comprise a number 2n, with n greater than or equal to 3 of groups of ducts in a number n of zones, each zone comprising two independent groups of ducts.

According to this option, each zone defined above corresponds to an axial portion of the balloon cuff that can be inflated or deflated independently of the adjacent portions.

The groups of independent ducts organized according to sections of the balloon cuff placed successively along the axis of the balloon cuff make it possible to implement a peristaltic movement of the balloon cuff. The outer surface of the balloon cuff can then be driven by a peristaltic wave movement propagating along the axis.

This is particularly the case if the balloon cuff comprises three subgroups of independent ducts in the second group.

Finally, according to one variant, there may be:
a main network of ducts covering the entire balloon cuff and ensuring an overall seal of the balloon cuff, for example a first group of ducts which have a main direction parallel to the axis and
a secondary network comprising a second group of ducts which have a main direction parallel to the circumferential direction, the second group being formed by three subgroups independently controllable according to zones of the balloon cuff successively placed along the axis of the balloon cuff.

For example, the primary network comprises longitudinal channels for deployment of the balloon cuff and the secondary network comprises circular channels for peristaltic pumping. The primary network and the secondary network do not communicate fluidly with each other.

### Tracheal Intubation Device

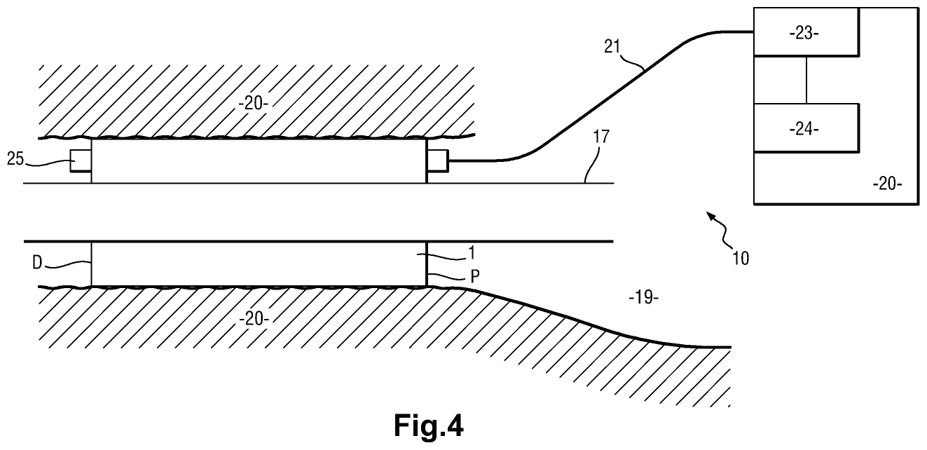

According to a second aspect, and with reference to FIG. 4, the invention relates to a device 10 configured to form an artificial airway at the trachea 19 of a patient, called a tracheal intubation device.

The trachea 19 is defined in the patient's esophagus 20.

The intubation device 10 comprises:
an artificial respiration tube 17,
a balloon cuff 1 as previously presented.

The balloon cuff 1 is mounted on the artificial respiration tube 17 so that the inner surface surrounds the tube. The central axis of the balloon cuff then corresponds to the axis of the tube.

The tube 17 of the tracheal intubation device 10 may comprise either an endotracheal tube, a tracheotomy cannula or a tracheostomy cannula.

Such a device 10 can be used to implement spontaneous respiration, i.e., without a mechanical ventilation module.

Such a device can be used to implement artificial respiration, i.e., with a mechanical ventilation module that fixes the flow of fluid through the artificial respiration tube.

In this second case, the device comprises a mechanical ventilation module 20 comprising:
a reservoir 24 of fluid under pressure connected to the ducts of the balloon cuff,
a control device 23 which controls the fluid reservoir 24 to apply a particular pressure to the fluid so that the balloon cuff 1 can be placed in the deflated or inflated state, and
a fluid connection 21 between the control device 23 and the balloon cuff.

When the balloon cuff 1 comprises several groups of ducts whose pressure can be adjusted independently, the control device 23 is fluidly connected independently to each group of ducts.

In a particular embodiment, the intubation device further comprises a pressure sensor 25 placed downstream of the balloon cuff with regard to the cranio-caudal direction, i.e., once the balloon cuff has been placed in the trachea, between the balloon cuff and the patient's lungs.

In other words, once the balloon cuff has been placed in the trachea, the balloon cuff is placed between the pressure sensor 25 and the patient's mouth.

The pressure sensor 25 can be configured to be placed after the balloon cuff is placed in the trachea:
against the distal end D of the balloon cuff, or
against an inner wall of the trachea, or
at the distal end of the tube, i.e., at the lung end of the tube,
or even between the outer surface of the tube and the inner surface of the balloon cuff.

The pressure sensor 25 is configured to acquire a measurement of excess pressure in the trachea.

The excess pressure may be representative of a coughing effort by the patient.

The pressure sensor 25 is connected to the control device 23 which controls the reservoir of fluid under pressure so that the excess pressure measurement is conveyed to the mechanical ventilation module 20.

The pressure sensor 25 may be a piezoelectric type sensor.

### Method for Controlling the Tracheal Intubation Device-Peristaltic Movement

According to a third aspect, a method of controlling a tracheal intubation device is proposed.

In a first step, the intubation device 10 is prepared to be inserted into the patient's trachea.

To this end, the balloon cuff 1 is mounted on the tube 17 of the device 10. The control device 23 is adjusted so that the balloon cuff 1 is configured in its deflated state.

Optionally, a pressure sensor 25 is disposed downstream of the balloon cuff with regard to the cranio-caudal direction projecting from the balloon cuff on the side of the distal end D of the balloon cuff.

The balloon cuff 1 mounted on the tube 17 is, in a second step, inserted into the trachea 19 of the patient.

Once the device 10 has been placed at a suitable location, the control device 23 is adjusted in a third step so that the balloon cuff is configured in its inflated state.

The balloon cuff as described up to now also makes it possible to cause specific movements of the structure from the injected fluid as a function of variables such as the flow rate, volume or pressure over time of this injected fluid. The induced movement may correspond to either an injection or a withdrawal of the fluid from the ducts. The movement may also correspond to an alternation between injection and withdrawal of fluid in the ducts.

When the balloon cuff comprises different groups of independently controllable organized channels distributed in successive zones along the axis, the control method may also comprise a movement of the balloon cuff surface according to at least one progressive wave along the axis of the balloon cuff.

Numerous variations in the method are possible in terms of shapes and movements to control the outer surface of the balloon cuff.

In one example, the balloon cuff comprises
a first group of ducts having a main direction parallel to the axis, and
a second group of ducts having a main direction parallel to the circumferential direction.

These two groups are independently controlled regarding their pressurization by the injected fluid. In other words, the fluid can be injected into the ducts of a first group without injecting the fluid into the ducts of the second group.

The first group especially ensures the deployment of the balloon cuff and the sealing between the upstream and downstream of the balloon cuff.

These two groups enable the outer surface to be moved by injecting fluid into the first group of ducts and by injecting fluid into the second group of ducts, so as to modify an inflated state of the outer radial surface.

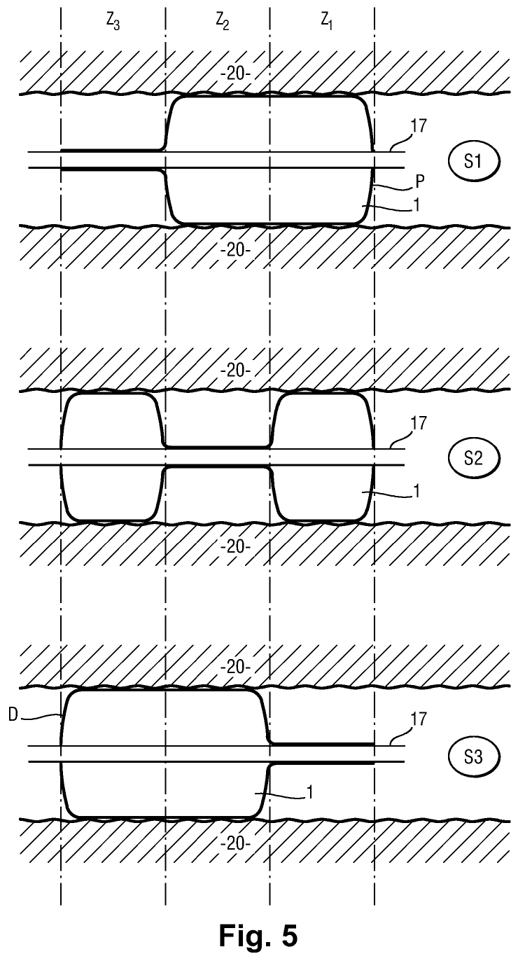

In reference to FIG. 5, the second group of balloon cuff ducts comprises three subgroups of ducts independently controllable with regard to the injection of the fluid: a proximal subgroup in the proximal zone Z1, a central subgroup in the central zone Z2, and a distal subgroup in the distal zone Z3, as described above. The following three substeps illustrate a possible example of variation.

In a first substep, illustrated S1 in FIG. 5, the control device is adjusted so that the balloon cuff is configured in a first transition state in which the distal subgroup in zone Z3 of ducts is deflated and the central subgroup and the proximal subgroup in zones Z1 and Z2 are inflated.

In a second substep, illustrated S2 in FIG. 5, the control device is adjusted so that the balloon cuff is configured in a second transition state in which the central subgroup in zone Z2 of ducts is deflated and the distal subgroup and the proximal subgroup in zones Z1 and Z3 are inflated.

In a third substep, illustrated S3 in FIG. 5, the control device is adjusted so that the balloon cuff is configured in a third transition state in which the proximal subgroup of ducts in zone Z1 is deflated and the central subgroup and the distal subgroup in zones Z2 and Z3 are inflated.

Such movement produces a transit or transfer of bronchial secretions from the distal face of the balloon cuff to the proximal face and the nasopharynx.

This at least partially spares the patient from the dangerous and painful procedure of manually aspirating the secretions.

The movement induced on the surface of the balloon cuff has the specific feature of inducing a transit or transfer of tracheal secretions by having an internal/external orientation that allows them to be brought above the balloon cuff where they will then be easily aspirated without harming the patient's ventilation. The balloon cuff retains its seal despite the peristaltic movement: There is always a portion of the outer surface of the balloon cuff in contact with the trachea, regardless of the phase of the movement.

It is possible to configure the mechanical ventilation module 20 so that this movement of the outer surface occurs at a regular rate which corresponds to the flow of pulmonary secretions generated by the patient. This flow varies from patient to patient and depends largely on the patient's lung function. It also depends on the patient's specific disorder and can change over time. It will therefore be adapted over time for the same patient.

When the device comprises a pressure sensor, the monitoring method may comprise detection of an excess pressure in the trachea.

When this is detected by the sensor, the information is communicated to the control device which then triggers the steps of the peristaltic movement.

The evacuation of bronchial secretions is then greater since the natural phenomenon of coughing in the trachea is synchronized with the movement of the outer surface of the balloon cuff.

It is possible to configure the mechanical ventilation module 20 so that this movement of the outer surface presents a result of the movements implemented at zero sum in the sense that the movement of the outer surface does not induce displacement of the balloon cuff structure or of the tube with regard to the trachea.

1. A balloon cuff for a tracheal intubation device,
the balloon cuff comprising inner walls,
the inner walls defining ducts passing through the balloon cuff,
the balloon cuff being elastically deformable, the balloon cuff extending between an inner surface surrounding an axis and an outer surface surrounding the inner surface,
the balloon cuff being configured to transition from a deflated state to an inflated state by injection of a fluid into the ducts so that the outer surface moves away from the axis.

2. The balloon cuff according to claim 1, wherein the balloon cuff defines a width of the outer surface and a thickness between the inner surface and the outer surface, a ratio of the thickness, over the width of the outer surface is greater than 10% when the balloon cuff is in the deflated state.

3. The balloon cuff according to claim 1 wherein the balloon cuff defines a duct size for each duct, a ratio of the duct size over a closer distance between the duct and the outer surface is less than or equal to 100% when the balloon cuff is in the inflated state.

4. The balloon cuff according to claim 1, wherein the inner surface and the outer surface have cylindrical shapes defined by an axial direction parallel to the axis, the inner surface and the outer surface having at least partly circular cross-sections in a plane orthogonal to the axis.

5. The balloon cuff according to claim 1, wherein the ducts have a main direction parallel to the axis, the ducts preferably extending parallel to the axis from a proximal end of the balloon cuff to a distal end of the balloon cuff.

6. The balloon cuff according to claim 1, wherein the ducts have a main direction parallel to a circumferential direction with respect to the axis, the ducts.

7. The balloon cuff according to claim 1 comprising groups of independently controllable ducts, the groups being located in sections of the balloon cuff, the sections being contiguous in pairs and placed successively along the axis.

8. The balloon cuff according to claim 1, wherein the balloon cuff defines a thickness between the inner surface and the outer surface, the thickness being less than or equal to 1 mm when the balloon cuff is configured in the deflated state.

9. The balloon cuff according to claim 1, the balloon cuff being formed predominantly of synthetic polymer material.

10. A tracheal intubation device configured to form an artificial airway at the patient's trachea, the intubation device comprising:
an artificial respiration tube,
a balloon cuff according to claim 1, the balloon cuff being mounted on the artificial respiration tube, so that the inner surface surrounds the tube.

11. The tracheal intubation device according to claim 10, wherein the intubation device comprises a pressure sensor downstream of the balloon cuff with regard to the cranio-caudal direction, the pressure sensor being configured to acquire a pressure measurement representative of a coughing effort by the patient.

12. A method of controlling a tracheal intubation device, the tracheal intubation device being in accordance with claim 10, the method comprising setting the outer surface in motion by injecting fluid inside a first group of ducts and by injecting fluid inside a second group of ducts, so as to modify a state of inflation of the outer surface.

13. A method of control according to claim 12, the tracheal intubation device being taken according to claim 11, the method comprising measuring a pressure in the trachea, comparing the measured pressure and a reference pressure, triggering the step of setting the outer surface in motion based on the result of the comparison step.

14. The balloon cuff according to claim 2, wherein the ratio of the thickness, over the width of the outer surface is greater than 20% when the balloon cuff is in the deflated state.

15. The balloon cuff according to claim 3, wherein the ratio of the duct size over the closer distance between the duct and the outer surface is less than or equal to 50%.

16. The balloon cuff according to claim 4, wherein the outer surface comprises a flat area, the flat area extending along the entire outer surface in the axial direction, the flat area being parallel to the axis.

17. The balloon cuff according to claim 6, wherein the ducts extend in an arc of a circle around the axis.

18. The balloon cuff according to claim 8, wherein the thickness being less than or equal to 0.5 mm when the balloon cuff is configured in the deflated state.

19. The balloon cuff according to claim 9, the synthetic polymer material, being silicone or polyurethane.
